(19) United States
(12) Patent Application Publication
(10) Pub. No.: US 2006/0045919 A1
(43) Pub. Date: Mar. 2, 2006

(54) Homeopathic compositions and methods for the treatment of cancer

(75) Inventor: Wayne B. Jonas, Alexandria, VA (US)

Correspondence Address: SPECKMAN LAW GROUP PLLC, 1501 WESTERN AVE, SEATTLE, WA, 98101, US

(73) Assignee: SAMUELI INSTITUTE, Alexandria, VA (US)

(21) Appl. No.: 10/928,506

(22) Filed: Aug. 27, 2004

(51) Int. Cl. A61K 36/185; A61K 36/13; A61K 35/12; A61K 33/24; A61K 33/38; A61K 33/32

(52) U.S. Cl. 424/520; 424/641; 424/617; 424/618; 424/726; 424/725; 424/770

(57) ABSTRACT

Provided are homeopathic compositions and methods for the preparation and use of such homeopathic compositions. Therapeutic methods comprise the administration of combinations of homeopathic compositions to provide the effective treatment of cancer, such as prostate cancers. Homeopathic compositions may be prepared by extracting from a patient a tissue sample comprising a tumor cell and a reactive tissue cell that is located in proximity to the tumor cell. Such a tissue sample is suspended in a liquid carrier solution and succussed serially in a liquid carrier solution to provide the homeopathic composition having a homeopathic potency. Homeopathic methods comprise the administration of a homeopathic composition optionally in combination with a second homeopathic composition. Exemplary second homeopathic compositions include Thuja occidentalis, Conium maculaturm, and Sabal serulata.

### BACKGROUND OF THE INVENTION

1. Technical Field of the Invention

The present invention relates generally to the treatment of cancer. More specifically, provided herein are homeopathic compositions as well as methods for the preparation and use of homeopathic compositions that are useful for the treatment of cancers, including prostate cancers.

2. Description of the Related Art

In recent years, there has been a dramatic increase in the use of non-allopathic compositions and therapeutic regimens for the treatment of cancers—referred to generally as complementary and alternative medicines (CAM). Use of alternative medical approaches and medicines, such as homeopathy, is widespread.

Homeopathy dates back to the eighteenth century and is founded on the principle of pharmacology and biology that “like cures like.” Jonas et al., Ann. Intern. Med. 138:393-399 (2003). In 1877, Hugo Schultz postulated that the effect of a stimulus on a living cell is indirect and proportional to its intensity and quantity. Later, in 1888, he demonstrated that very low concentrations of yeast toxins increased yeast growth over 100 fold. Concurrently, the psychiatrist Rudolph Arndt developed his “Basic Law of Biology,” which provides that weak stimuli slightly accelerate the vital activity while middle-strong stimuli raise, strong stimuli suppress, and very strong stimuli halt vital activity. These separate observations were formulated by Arndt in 1888 into one of the earliest laws of pharmacology representing the homeopathic effect, the Arndt-Schultz law wherein every stimulus on a living cell is said to elicit an activity that is inversely proportional to the intensity of the stimulus. Martius, Muench. Med. Wschr. 70(31):1005-1006 (1923). This law was later restated by Hueppe as follows: for every substance, small doses stimulate, moderate doses inhibit, and large doses kill.

Allopathic medicine, with its emphasis on moderate drug doses, works to inhibit undesired physical symptoms and to kill undesired pathogens. Homeopathic medicine, in contrast, begins with small doses and moves towards progressively higher dilutions to stimulate the body's own natural electromagnetic forces. One of the basic tenets of homeopathic medicine is that a cure for a disease can be evoked by using a high dilution medicine that resembles, yet is distinct from, the cause of the disease. Thus, homeopathic practitioners often utilize extremely high dilutions of hormones or cells as a remedy to a disease.

Homeopathy is widely accepted as a useful therapeutic approach throughout Europe, the British Commonwealth countries, and India, and has been demonstrated to have characteristic and reproducible effects. Critical reviews of more than 100 controlled and/or clinical studies of homeopathy conclude that patients received positive healing benefits from homeopathy beyond the placebo effect. Jonas et al., Ann. Intern. Med. 138:393-399 (2003); Kleijnen et al., Bmj. 910418 302(6772):316-323 (1991); Linde et al., Lancet. 350(9081): 834-843 (1997); and Reilly et al., Lancet. 344:1601-1606 (1994).

While the precise mechanism of action of homeopathic medicines is unknown, magnetic resonance imaging on serial dilutions of substances indicates that the hydroxyl (OH) groups in the solvent of solutions continue to change as dilutions become successively higher. Sacks, J. Hol. Med. 5:172-177 (1983); Smith et al., J. Am. Inst. Hom. 61:197-212 (1968); and Smith, et al., J. Am. Inst. Hom. 59:263-280 (1966).

It has been postulated that highly dilute compounds transfer biological activity to cells by electromagnetic fields. Benveniste, Frontier Perspec. 3(2):13-15 (1993). Del Giudice et al. have hypothesized that interactions between the electric dipoles of water and the radiation fields of a charged molecule generate a permanent polarization of water that becomes coherent and has the ability to transmit specific information to cell receptors. Del Giudice et al., In Biological Coherence and Response to External Stimuli. 49-64 (Frohlich H, ed., Berlin: Springer-Verlag, 1988). It is evident that the specific effects of homeopathic preparations are of a physical, non-molecular origin, yet provide potent biological activities that are clinically effective.

A common principle of homeopathy is the “Law of Similars,” which is founded in the science of pharmacology and provides that a drug has two effects on the body, a direct effect and the subsequent reaction of the body to the drug, evoking symptoms or side effects. In homeopathy, as the active agent is diluted, some of the positive benefits of the active agent remain, and new characteristics of the active agent become available to the body to not only alleviate side effects, but also to ameliorate other symptoms the patient may have.

A critical review of the clinical literature in homeopathy demonstrates that homeopathic preparations have significant effects beyond a placebo. Homeopathic preparations of diseased tissue have been used in homeopathic practice and are referred to as nosodes. Such preparations have not been prepared for use by individual patients using the individual patient's tissue. Lesion tissue was used in combination with surrounding reactive tissue in homeopathic preparations in a study of tularemia infection. Jonas et al., J Sci. Explor. 14(1):35-52 (2000). There remains, however, a need in the art for improved homeopathic compositions and methods for the treatment of cancers, such as prostate cancer.

### SUMMARY OF THE INVENTION

The present invention addresses these and other related needs by providing, inter alia, homeopathic compositions as well as methods for the preparation and use of those compositions. Thus, provided herein are therapeutic regimens that employ inventive homeopathic compositions and are effective in the treatment of cancers, including but not limited to, prostate cancers.

The present invention is based, in part, on the observation that certain homeopathic compositions, as disclosed herein, are effective in increasing animal survival parameters in the art accepted Copenhagen rat model system for cancer. Based on the strong efficacy shown in this animal model system, it is contemplated that the homeopathic compositions and treatment regimens of the present invention may be advantageously employed in the treatment of human cancers including, but not limited to, human prostate cancers.

More specifically, and as described in further detail herein below, the presently disclosed homeopathic compositions that form the basis for the cancer treatment regimen are referred to collectively as “PROCAN” or “the PROCAN technology” and will find broad application in the treatment of solid prostate tumors.

Thus, within certain embodiments, the present invention provides methods for preparing PROCAN homeopathic compositions having a homeopathic potency, the methods comprising the steps of: (a) excising from a cancer patient a tissue sample comprising a tumor cell and a reactive tissue, wherein the reactive tissue is sampled from a location in proximity to the tumor cell; (b) suspending the tissue sample in a liquid carrier solution; and (c) diluting and succussing a portion of the suspended sample serially in the same or a different liquid carrier solution, thereby providing a homeopathic composition having a homeopathic potency.

Other embodiments of the present invention provide homeopathic compositions comprising: (a) a tumor cell; (b) a reactive tissue; and (c) a liquid carrier solution, wherein the homeopathic composition is diluted and succussed to a homeopathic potency of at least about 200 c. Alternatively, the homeopathic composition may be diluted and succussed to a homeopathic potency of at least about 500 c, at least about 750 c, or at least about 1000 c.

Within still further embodiments, the present invention provides homeopathic compositions prepared by a process comprising the steps of: (a) excising from a cancer patient a tissue sample comprising a tumor cell and a reactive tissue, wherein the reactive tissue is taken from a location in proximity to the tumor cell; (b) suspending the tissue sample in a liquid carrier solution; and (c) diluting and succussing a portion of the suspended sample serially in the liquid carrier solution, thereby providing a homeopathic composition having a homeopathic potency.

Yet other embodiments of the present invention provide methods for prolonging the survival of an animal afflicted with a cancer. These methods comprise the step of administering to the animal a homeopathic composition as described above and in further detail herein below. Alternative related embodiments of the present invention provide methods for the treatment of cancer in a patient in need thereof. These methods comprise the step of administering to the patient a homeopathic composition as summarized above and as described in further detail herein below. Most typically, the patient is a human patient.

PROCAN preparations having a homeopathic potency of at least about 100 c are suitable for many applications, and PROCAN preparations having a homeopathic potency of 1000 c are preferred for many applications. Other suitable potencies are 30 c, 500 c, 800 c and occasionally the mother tincture. The homeopathic composition is generally prepared in a liquid carrier solution and may thereafter be prepared in various delivery forms, including liquid delivery forms, solid delivery forms such as tablets or other solid carriers on which the liquid is deposited, and other oral delivery forms, including time release formulations, as well as transdermal and other delivery forms, including IV, sub-dernal, and intramuscular injections.

Within certain aspects of the compositions and methods of the present invention, the tumor cell may be from a tumor selected from the group consisting of soft tissue sarcomas, lymphomas, and tumors of the brain, esophagus, uterine cervix, bone, lung, endometrium, bladder, breast, larynx, colon/rectum, stomach, ovary, pancreas, adrenal gland and prostate. Other tumors may also be utilized.

Within other aspects of these compositions and methods, the reactive tissue is selected from the group consisting of monocytes, T cells, cytokines (including TNF-a, IL-12, IP-10, MIP-2, IFN-g, MCP-5, IL-1, RANTES, and IL-6), cell membranes, inflammatory cells, and other mediators.

Typically, the liquid carrier solution employed in the compositions and methods presented herein is an aqueous solution and generally comprises an alcohol and water such as, for example, ethanol (EtOH in water). Most typically, the liquid carrier solution comprises at least about 60% EtOH. Alternatively, the liquid carrier solution may comprise at least about 70% EtOH, at least about 80% EtOH, at least about 90% EtOH or 100% water.

Other aspects of the methods presented herein further comprise the step of administering the PROCAN homeopathic composition having a homeopathic potency in combination with one or more additional homeopathic preparations. Within such aspects, the one or more other homeopathic composition(s) may be selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium.

Depending on the specific treatment regimen contemplated, the homeopathic compositions that are especially suitable for use in combination with PROCAN for treating prostate cancer may be selected from the group consisting of Thuja occidentalis, Conium maculaturm, and Sabal serulata.

Most typically, the homeopathic potency of the second homeopathic composition(s) used in combination with PROCAN is at least about 100 c. Alternatively, the homeopathic potency of the second homeopathic composition may be at least about 200 c, or at least about 1000 c.

Within certain aspects of these methods, the homeopathic composition is administered at least twice. For example, the homeopathic composition may be administered to the animal at least once a week for at least about two weeks, at least about three weeks, or at least about four weeks.

Within still further aspects of these methods, the PROCAN homeopathic composition is administered at least once weekly and the second homeopathic composition is administered at least once weekly. Generally, but not necessarily, the second homeopathic composition is administered at a different time from the PROCAN homeopathic composition. Alternatively, the PROCAN homeopathic composition may be administered at least once weekly and the second homeopathic composition may be administered at least twice weekly.

### BRIEF DESCRIPTION OF THE FIGURES

FIG. 1A shows a Kaplan-Meier analysis of the survival rate of tumor free Copenhagen rats treated with PROCAN regimen.

FIG. 1B shows a Kaplan-Meier analysis of the survival rate of Copenhagen rats inoculated with MAT-LyLu cells and treated with PROCAN regimen.

FIG. 2A shows the effect of PROCAN regimen treatment on tumor volume in Copenhagen rats inoculated with MAT-LyLu cells.

FIG. 2B shows the effect of PROCAN regimen treatment on tumor weight in Copenhagen rats inoculated with MAT-LyLu cells.

FIG. 3 shows the effect of PROCAN regimen treatment on average body weight of Copenhagen rats inoculated with MAT-LyLu cells.

### DETAILED DESCRIPTION OF THE INVENTION

As indicated above, the present invention is based, in part, on the observation that certain homeopathic compositions, disclosed herein, are effective in increasing animal survival parameters in the art accepted Copenhagen rat animal model system for cancer. Homeopathic compositions and treatment regimens of the present invention may be advantageously employed in the treatment of cancers, including human cancers, such as, for example, human prostate cancers.

Exemplified herein are homeopathic compositions and methods for their preparation and use that are suitable for the treatment of cancers characterized by solid tumors, more specifically solid prostate tumors, that are referred to collectively as “PROCAN” or “the PROCAN technology.”

The above-mentioned and additional features of the present invention and the manner of obtaining them will become apparent, and the invention will be best understood by reference to the more detailed description, below, read in conjunction with the following definitions.

Definitions

As used herein, “tumor cells” that are suitably employed in the compositions and methods of the present invention include cells from one or more of the following tumors: soft tissue sarcomas, lymphomas, and tumors of the brain, esophagus, uterine cervix, bone, lung, endometrium, bladder, breast, larynx, colon/rectum, stomach, ovary, pancreas, adrenal gland and prostate. Cells from other tumors may also be used in the compositions and methods presented herein.

As used herein, the term “reactive tissue” refers to those cells and cellular components that are involved in the individual's reaction to the tumor. Typically, reactive tissue employed in the compositions and methods of the present invention include the following: monocytes, T cells, cytokines (including TNF-a, IL-12, IP-10, MIP-2, IFN-g, MCP-5, IL-1, RANTES, and IL-6), cell membranes, inflammatory cells, and other mediators.

As used herein, the term “succussing” refers to the mixing or shaking that is essential to the method of making a homeopathic remedy. Succussion is well known in the art and science of homeopathy. A preferred succussion methodology for use in the preparations of the present invention is to shake mixtures firmly from a height of 12 inches 120 times at a rate of two strokes per minute.

The homeopathic compositions of the present invention are prepared by the process of “potentization,” which comprises a succession of dilutions and succussions of the starting tumor tissue. For example, a tumor cell comprising tissue sample may be suspended in nine parts of a “liquid carrier solution,” most typically an aqueous solution such as distilled water or an alcohol solution, such as an ethanol (EtOH) solution, and the resulting mixture shaken vigorously, i.e. succussed. Typically, nine parts of the initial solution comprising tumor tissue in a liquid carrier solution may subsequently be diluted with nine parts of the liquid carrier solution and the resulting second diluted solution again subjected to vigorous succussion. This stage of dilution and succussion may be further repeated several times.

As used herein, the term “homeopathic potency” is defined in reference to the relative dilution of the starting tissue sample. The potency is defined in terms of a number, where the higher the number, the higher the dilution. 1000 c, for example, is more diluted (and thus, has a higher homeopathic potency) than 750 c, which in turn has a higher homeopathic potency than 500 c, which in turn has a higher homeopathic potency than 200 c.

Homeopathic Compositions and Methods for Preparing Homeopathic Compositions

The present invention provides homeopathic compositions having homeopathic potency as well as methods for preparing such homeopathic compositions. Homeopathic compositions disclosed herein comprise: (a) a tumor cell; (b) a reactive tissue; and (c) a liquid carrier solution, wherein the composition is diluted and succussed to a homeopathic potency of at least about 1000 c. Typically, such homeopathic compositions are prepared by a process comprising the steps of: (a) excising from a cancer patient a tissue sample comprising a tumor cell and a reactive tissue wherein the reactive tissue is proximal to the tumor cell; (b) suspending the tissue sample in a liquid carrier solution; and (c) diluting and succussing a portion of the suspended sample serially in the liquid carrier solution thereby providing a homeopathic composition having a homeopathic potency.

Homeopathic compositions of the present invention may be prepared using tissue samples from a wide variety of tumors including, for example, soft tissue sarcomas, lymphomas, and tumors of the brain, esophagus, uterine cervix, bone, lung, endometrium, bladder, breast, larynx, colon/rectum, stomach, ovary, pancreas, adrenal gland and prostate.

In the case of prostate tumors, as exemplified herein by PROCAN, individualized homeopathic compositions for treatment thereof may be formulated for each subject being treated by removing a sample of the individual's prostate tumor along with surrounding “reactive tissue” using, for example, a biopsy needle.

Typically, a tissue sample is obtained from a fresh biopsy in which at least 0.1 to 3.0 mm of tumor and 0.5 to 1.0 mm of reactive tissue is obtained. Samples may be obtained by needle biopsy or by open resection. It is important that the tumor sample used in the homeopathic compositions, such as PROCAN, include both tumor cells and reactive prostate tissue in proximity to the tumor.

Depending upon the nature of the tumor tissue employed, the reactive tissue may include monocytes, T cells, cytokines, cell membranes, inflammatory cells and mediators, and other cells and cellular components that are involved in the individual's reaction to the tumor.

Following excision of the tissue sample, the removed tumor and proximal reactive tissue sample is suspended in a carrier solution such as an alcohol and/or aqueous solution then macerated or homogenized. Typically, liquid carrier solutions comprise a mixture of an alcohol and water. For example, in the homeopathic compositions presented herein, the liquid carrier solutions comprise a mixture of ethanol (EtOH) and water. Most commonly, liquid carrier solutions are at least about 60% EtOH in water (volume to volume), at least about 70% EtOH in water, at least about 80% EtOH in water, or at least about 90% EtOH in water.

The ratio of tissue sample to solution is, most commonly, not greater than 1:100 (weight to volume). A particularly suitable preparation comprises 2-3 grams of tissue in 20 ml of solution. The tumor sample may be homogenized in a 60% ethanol solution, for example, by mixing in a blender for 5 minutes. A portion of the homogenate is then formulated, in a 1:99 proportion, with additional liquid carrier solution, such as in 60% ethanol liquid carrier solution, and succussed, to provide a 1 c PROCAN formulation. The PROCAN formulation is preferably succussed repeatedly, manually or using an automated system, or using a combination of manual and automated succussion techniques, to produce a homeopathic 1000 c PROCAN preparation.

The PROCAN homeopathic preparation formulated from the tumor and reactive tissue sample extracted from an individual patient and administered to that individual patient, according to a prescribed regimen, form the basis for the prostate cancer treatment of the present invention. Because each PROCAN preparation is prepared from a cell sample taken from individual subjects, each treatment is specific to the individual being treated.

Methods of Treatment Employing Homeopathic Compositions

As noted above, the present invention provides methods of treatment that employ one or more homeopathic composition. Thus, within certain embodiments, the present invention provides methods for prolonging the survival of an animal afflicted with a cancer. The animal may be a human. These methods comprise the step of administering to the animal a homeopathic composition as described above and in further detail herein below. Alternative embodiments of the present invention provide methods for the treatment of cancer in a patient in need thereof. These methods comprise the step of administering to the patient a homeopathic composition as summarized above and as described in further detail herein below. Most typically, the patient is a human patient.

Within certain aspects of these methods, the homeopathic composition is administered at least twice. For example, the homeopathic composition may be administered to the animal at least once a week for at least about two weeks, at least about three weeks, or at least about four weeks.

Other aspects of these methods further comprise the step of administering a second homeopathic composition. Most typically, the second homeopathic composition is selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium. Depending on the specific treatment regimen contemplated, the second homeopathic composition may be selected from the group consisting of Thuja occidentalis, Conium maculaturm, and Sabal serulata.

Most typically, the second homeopathic composition is added to a homeopathic potency of at least about 200 c. Alternatively, the homeopathic potency of the second homeopathic composition may be added to a homeopathic potency of at least about 500 c, at least about 750 c, or at least about 1000 c.

The homeopathic composition may be administered at least once weekly and the second homeopathic composition is administered at least once weekly. Alternatively, the homeopathic composition may be administered at least once weekly and the second homeopathic composition may be administered at least twice weekly.

According to one treatment regimen of the present invention, a homeopathic composition, exemplified herein by a PROCAN composition, is administered to a patient orally at least once weekly. Typically, for administration to a human, the dosage will be approximately 10 ml/week. In other treatment regimens, the PROCAN preparation may be administered as frequently as daily, and as infrequently as once.

One or more additional homeopathic compositions may be administered alternately with or in addition to the PROCAN preparation. Exemplary such additional homeopathic compositions may be selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium. The one or more additional homeopathic preparations are preferably selected from the group consisting of Thuja occidentalis, Conium maculaturm, and Sabal serulata. In one embodiment, two of the three homeopathic preparations selected from the group consisting of Thuja occidentalis, Conium maculaturm, and Sabal serulata are administered alternately with the PROCAN preparation and, in an especially preferred embodiment, each of the three homeopathic preparations—Thuja occidentalis, Conium maculaturm, and Sabal serulata—is administered alternately with the PROCAN preparation.

The treatment regimen and homeopathic potencies of Thuja occidentalis, Conium maculaturm, and Sabal serulata may vary depending on the individual patient's symptoms and tumor type, but generally are greater than about 200 c, greater than about 500 c, greater than about 750 c, or greater than about 1000 c. Typical homeopathic potencies are as follows: Thuja occidentalis (1000 c), Conium maculaturm (1000 c) and Sabal serulata (200 c). Also contemplated are treatment regimens comprising one or more additional remedy including, but not limited to, hydrastis and/or lycopodum.

In one such exemplary embodiment, at least one dosage of each of the homeopathic preparations Thuja occidentalis, Conium maculaturm, and Sabal serulata is administered to a patient between PROCAN administrations. In another embodiment, homeopathic preparations selected from the group consisting of Thuja occidentalis, Conium maculaturm, and Sabal serulata are each administered at least once weekly between PROCAN administrations. In yet another embodiment, homeopathic preparations selected from the group consisting of Thuja occidentalis, Conium maculaturm, and Sabal serulata are each administered at least twice weekly between PROCAN administrations.

In one treatment regimen, homeopathic preparations of Thuja occidentalis, Conium maculaturm, and Sabal serulata are administered to a subject, serially, between PROCAN administrations. In another treatment regimen, homeopathic preparations of Thuja occidentalis, Conium maculaturm, and Sabal serulata are administered to a subject, once daily, on all days between weekly PROCAN administrations in a manner such that Thuja occidentalis is administered on the first and fourth days of the week; Conium maculatum is administered on the second and fifth days of the week; Sabal serulata is administered on the third and sixth days of the week; and PROCAN is administered on the 7^{th} day of the week.

In an especially preferred treatment regimen that demonstrated efficacy in treating prostate cancers in an experimental mammalian model, homeopathic preparations of Thuja occidentalis, Conium maculaturm, and Sabal serulata were administered to the subject, once daily, on all days between weekly PROCAN administrations in a manner such that Thuja occidentalis (1000 c) was administered on the first and fourth days of the week; Conium maculatum (1000 c) was administered on the second and fifth days of the week; Sabal serulata (200 c) was administered on the third and sixth days of the week; and PROCAN (1000 c) was administered on the 7^{th} day of the week.

This treatment may be administered to a subject for at least 2 weeks and, in some embodiments, may persist until no detectable cancer cells remain in the subject's body. Thus, treatment may continue for four weeks to 2 years, or longer.

Kits Comprising Homeopathic Compositions

The present invention also contemplates treatment kits comprising the treatment agent(s) of the present invention. A treatment kit comprising PROCAN (1000 c) is provided, and may be supplemented with homeopathic preparations of Thuja occidentalis, Conium maculaturm and Sabal serulata. In one embodiment, a treatment kit of the present invention comprises PROCAN (1000 c), Thuja occidentalis (1000 c), Conium maculaturm (1000 c) and Sabal serulata (200 c).

### EXAMPLES

The following Examples demonstrate the efficacy of homeopathic compositions and methods of the present invention. The efficacy of the PROCAN homeopathic compositions and treatment regimens was demonstrated against prostate cancer induced by the administration of MAT-LyLu cells to Copenhagen rats. MAT-LyLu cells are an art accepted and routinely employed cell line that induces prostate cancer in mammals when administered in vivo. Based on these experimental results, using this animal system, it is contemplated that similar efficacy and treatment protocols will demonstrate efficacy against human tumors and, in particular, human prostate tumors. These Examples are offered by way of illustration not limitation.

### Example 1

### The Copenhagen Rat Animal Model System for Prostrate Cancer

This Example discloses the art accepted Copenhagen rat animal model system for prostate cancer.

MAT-LyLu cells were obtained from the American Type Culture Collection (ATCC; Manassas, Va.) and maintained in RPMI 1640 medium supplemented with 250 nM dexamethasone, 90%; fetal bovine serum, stored in a humidified incubator containing 5% CO₂ at 37° C. Male Copenhagen rats were first inoculated with MAT-LyLu cells (10,000 cells in 100 ml PBS) by intradermal injection into the rear right leg. The MAT-LyLu cells were allowed to grow in the rats for 7 days. By the 7^{th} day, solid tumors of approximately 1 cm in size were apparent, and reactive and inflammatory responses were well established in the rats.

### Example 2

### Preparation of Tissue Samples and Exemplary Homeopathic Compositions

This Example demonstrates the preparation of an exemplary PROCAN homeopathic composition suitable for the treatment of prostate cancer.

Tissue samples were obtained by excising the tumors and about 3 mm of surrounding tissue, including reactive tissue. The tissue samples were homogenized in a blender for 5 minutes with 20 ml of 60% ethanol. One part of this homogenate was mixed with 99 parts of 60% ethanol and shaken firmly from a height of 12 inches 120 times, at a rate of two strokes per minute. The product of this succussion was referred to as the PROCAN 1 c preparation. The dilution and succussion procedure was repeated and the PROCAN 1 c preparation was diluted 1:99 two additional times to produce a PROCAN 3 c preparation. PROCAN 3 c was further diluted and succussed, using an automated system, until a 1000 fold (1000 c) dilution was produced. The PROCAN 1000 c homeopathic preparation was used in the treatment regimen.

### Example 3

### Administration of a PROCAN Homeopathic Composition Increases Survival Duration in the Copenhagen Rat Animal Model System for Prostate Cancer

This Example demonstrates that administration of a PROCAN homeopathic composition of the present invention is effective in increasing the survival of Copenhagen rats afflicted with prostate cancer through the in vivo administration of MAT-LyLu cells.

Each of 98 4 to 5 week old Copenhagen rats was inoculated with 10,000 MAT-LyLu cells in 100 ml PBS by intradermal injection. The 98 inoculated rats (subjects) were randomly distributed into two groups of 49—one control and one treatment group. The control group received an equal volume of succussed water as a control and in place of the treatment(s). The investigators were blinded to the treatment status of the rats during the experiment. Both groups were similarly handled. All animals were housed in individual cages and had ad libitum access to food and water. The body weight of each of the animals was measured weekly.

Beginning on the day following inoculation with MAT-LyLu cells, each subject in the treatment group was inoculated with 100 μl of homeopathic remedy once daily by oral lavage according to the following schedule: first and fourth days: Thuja occidentalis (1000 c); second and fifth days: Conium maculatum (1000 c); third and sixth days: Sabal serulata (200 c); seventh day: PROCAN (1000 c). Subjects in the control group received equal volumes of succussed water on the same schedule. This treatment protocol was followed for ten (10) weeks following inoculation.

Tumors for subjects in both groups were measured every fourth day with calipers and tumor volume was calculated based on the measurements. The formula used for measuring the tumor volume (V) was 0.5236*a*b*c where a, b and c were the three radii (Gleave et al., 1991). At the end of the experiment, rats were sacrificed and blood was collected and centrifuged. The tumors and the lungs of the rats were removed, weighed and utilized for histological studies. The lungs were inspected for visible morphological changes. All tissues collected at the sacrifice were fixed in neutral buffered 10% formalin solution. The lungs were inflated with formalin in situ before fixing with formalin. The tissue was embedded in paraffin and 5 mm thick sections were obtained. The sections were then stained with hematoxylin-eosin staining. The pathologist who carried out the histological examinations was not aware of any treatment details. Tissue sections were visualized and captured using Nikon Eclipse E400 microscope attached with Nikon digital camera DXM1200.

In this Example, 1 animal from the untreated control group and 2 animals from the treatment group died on the 1st day after inoculation of MAT-LyLu cells. Inoculation of MAT-LyLu cells resulted in induction of tumors in 100% of untreated Copenhagen rats. The sizes of the tumor were measured every 4th day once the tumors attained measurable sizes after the onset of tumor. The averages of the measurements were calculated for each treatment group where the rats survived for at least 3 weeks. For the rats that had early end points, the last observed value before sacrifice was used at subsequent time points.

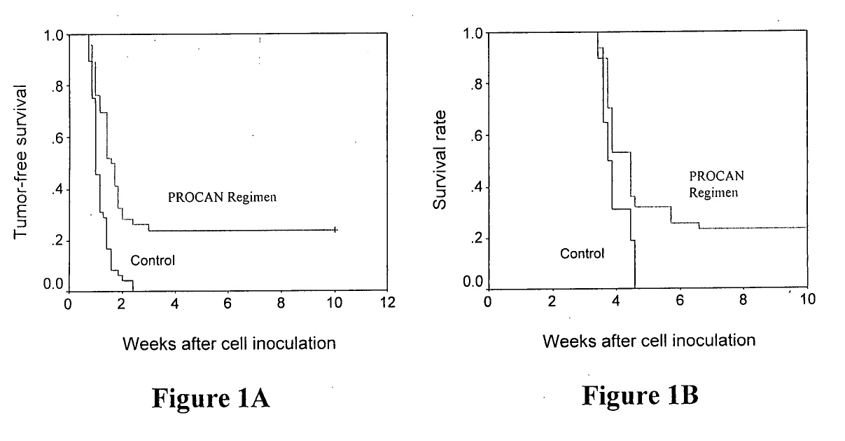

The Kaplan-Meier method was used to analyze the disease free survival and survival rates; results are shown in FIGS. 1A and 1B. The results demonstrated that tumors appeared earlier in the control group than the homeopathic treatment group. The median time for the first appearance of tumor was 7 days in the untreated control group and 11 days in the homeopathic treatment group. Mean weekly body weight and geometric mean tumor volume (every 4 days) were compared using repeated measures analysis of variance, followed by Bonferroni-adjusted post-hoc comparisons where appropriate. Average tumor weight at time of sacrifice was compared using 2-sided student's t test for independent samples, and the number of animals with visible lung nodules was compared using Fisher's exact test. P-values less than 0.05 were considered statistically significant.

The PROCAN 1000 c treatment regimen resulted in 23% decrease in tumor incidence compared to tumor incidence in control animals, with tumors occurring in only 36 of 47 animals. The tumor free survival curves differed significantly based on the log rank test (long rank statistic 22.64, P<0.0001). As the survival curves shown in FIG. 1B demonstrate, the tumor free rats remained disease free until the termination of the experiment (10 weeks from cell inoculation). The untreated rats needed to be sacrificed as the tumors grew very large, causing animals to lose weight, or become immobile and sick. The tumors in some rats were ulcerated and they were also sacrificed before the end of the experiment. The median time between inoculation and sacrifice was 26 days in the control group and 31 days in the treatment group. The p-value (0.0002) for the log rank test was highly significant.

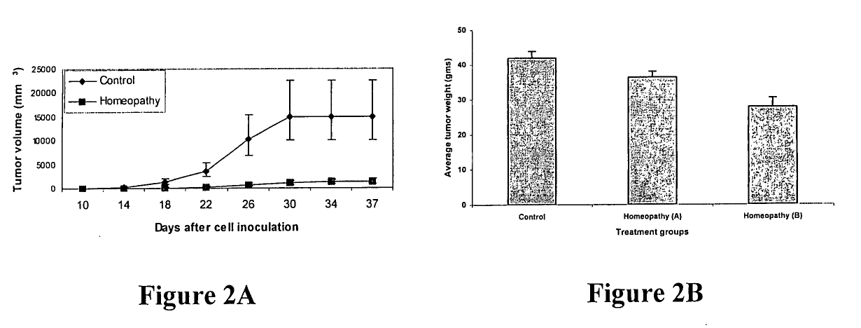

As shown in FIG. 2A, a decrease in tumor burden was observed in rats treated using the homeopathic PROCAN regimen compared to the untreated controls. After 3 weeks of tumor initiation, a significant increase of tumor growth was observed in the controls with a wide range of tumor sizes. PROCAN 1000 c regimen treatment inhibited tumor growth and showed a plateau in the growth rate. The rats that received PROCAN 1000 c regimen showed a 50% reduction in the mean tumor volume after 4 weeks of inoculation compared to untreated control group (P<0.001). This result supports the anti-tumor effect of homeopathic treatment.

Tumor weights were also measured at the time of sacrifice, and the tumor volume calculated for both treatment and control sample groups. The results demonstrated significantly lower average tumor weights for the PROCAN 1000 c treatment regimen group compared to the untreated group (p=0.001). The decrease in average tumor weight was 14 grams (approximately 33%) over the control group. Also, when the tumor weights only were compared between tumor bearing rats from the treatment and control regimens, there was a 5.5 gram difference (p=0.044) and approximately 13% reduction in tumor weights for the treated group compared to the control group. Untreated rats, which often had to be sacrificed earlier than the treated group, had higher average tumor weights than the treated groups.

The visible lung metastases in the tumor bearing rats showed a considerable variation between the PROCAN 1000 c regimen treated groups and untreated groups. In the treated group, 5.7% of the rats had visible lung metastases, while 10.4% of the rats in the untreated groups had metastases. However, the difference was not statistically significant (p=0.693).

### Example 4

### Administration of a PROCAN Homeopathic Composition is not Toxic in the Copenhagen Rat Animal Model System for Prostate Cancer

This Example demonstrates that administration of a PROCAN homeopathic composition of the present invention is not toxic to Copenhagen rats afflicted with prostate cancer though the in vivo administration of MAT-LyLu cells.

Example 4 followed the same treatment and control protocols as Example 3, using a sample of 20 rats, with a 10-individual control group and a 10-individual treatment group. All rats were inoculated with 10,000 MAT-LyLu cells in 100 ml PBS by intradermal injection. The 20 rats were then randomly distributed into two groups of 10 rats in each group. One group was the control group and received succussed water rather than a homeopathic treatment. The treatment group received the treatment described above in Example 3. The weekly schedule was followed until the 10^{th} week after cell inoculation, which was the end of the experiment. The tumor-free survival and survival rate results were similar to the results obtained in Example 3.

Two additional groups of Copenhagen rats were included in the two treatment and control groups described above to compare the effect of the PROCAN homeopathic treatment on the well being of tumor induced animals, versus the well being of animals in which tumors were not induced and that were either completely untreated (no placebo), or treated using the PROCAN homeopathic treatment. Four groups were tested: Group 1: normal control—no tumor induction, no treatment, no placebo; Group 2: Mat-LyLu tumor induced—placebo; Group 3: no tumor induction, PROCAN homeopathic treatment as described in Example 3; Group 4: Mat-LyLu tumor induced—PROCAN homeopathic treatment as described in Example 3. Average body weights were measured weekly for five weeks following inoculation.

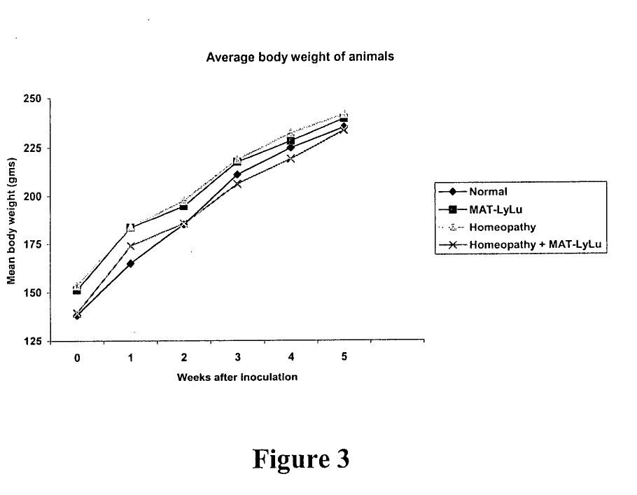

FIG. 3 shows the changes of mean body weight of the rats in different groups. There were no significant differences in body weight between groups. Interestingly, the rats in all groups had substantially the same mean body weight at the end of the five week period. None of the rats in any groups showed visible toxicity as measured by loss of appetite, hair loss or lack of movement, and that there were no significant differences in body weight between groups

In another set of measurements, serum testosterone levels were found to be down-regulated in untreated tumor bearing animals compared to normal control animals. However, homeopathy treated animals showed an increase in serum testosterone level.

What is claimed is:

1. A method for preparing a homeopathic composition having a homeopathic potency, said method comprising the steps of:
(a) excising from a cancer patient a tissue sample comprising a tumor cell and a reactive tissue wherein said reactive tissue is in proximity to said tumor cell;
(b) suspending said tissue sample in a liquid carrier solution; and
(c) diluting and succussing a portion of said suspended sample serially in said liquid carrier solution thereby providing a homeopathic compositions having a homeopathic potency.

2. The method of claim 1 wherein said tissue sample is excised from a tumor selected from the group consisting of a soft tissue sarcoma, a lymphoma, a brain tumor, an esophagus tumor, a uterine cervix tumor, a bone tumor, a lung tumor, an endometrial tumor, a bladder tumor, a breast tumor, a larynx tumor, a colon/rectum tumor, a stomach tumor, an ovary tumor, a pancreas tumor, an adrenal gland tumor, and a prostate tumor.

3. The method of claim 1 wherein said reactive tissue is selected from the group consisting of a monocyte, a T cell, a cytokine, a cell membrane, an inflammatory cell, and a mediator.

4. The method of claim 1 wherein said liquid carrier solution comprises at least 60% EtOH in water (volume to volume).

5. The method of claim 1, further comprising the step of adding to said homeopathic composition having a homeopathic potency a second homeopathic composition.

6. The method of claim 5 wherein said second homeopathic composition is selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium.

7. The method of claim 1 wherein the potency of said second homeopathic composition is added to a homeopathic potency of at least 200 c.

8. A homeopathic composition prepared by a process comprising the steps of:
(a) excising from a cancer patient a tissue sample comprising a tumor cell and a reactive tissue wherein said reactive tissue is proximal to said tumor cell;
(b) suspending said tissue sample in a liquid carrier solution; and
(c) diluting and succussing a portion of said suspended sample serially in said liquid carrier solution thereby providing a homeopathic compositions having a homeopathic potency.

9. The homeopathic composition of claim 8 wherein said tumor is selected from the group consisting of a soft tissue sarcoma, a lymphoma, a brain tumor, an esophagus tumor, a uterine cervix tumor, a bone tumor, a lung tumor, an endometrial tumor, a bladder tumor, a breast tumor, a larynx tumor, a colon/rectum tumor, a stomach tumor, an ovary tumor, a pancreas tumor, an adrenal gland tumor, and a prostate tumor.

10. The homeopathic composition of claim 8 wherein said reactive tissue is selected from the group consisting of a monocyte, a T cell, a cytokine, a cell membrane, an inflammatory cell, and a mediator.

11. The homeopathic composition of claim 8 wherein said liquid carrier solution comprises at least 60% EtOH in water (volume to volume).

13. The homeopathic composition of claim 8 wherein said homeopathic composition has a potency of at least 200 c.

14. The homeopathic composition of claim 8 having a homeopathic potency of at least 1000 c.

15. The homeopathic composition of claim 8 wherein said process comprises the further step of adding to said homeopathic composition a second homeopathic composition.

16. The homeopathic composition of claim 15 wherein said second homeopathic composition is selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium.

17. The homeopathic composition of claim 15 wherein said second homeopathic composition has a homeopathic potency of at least 200 c.

18. A homeopathic composition, comprising:
(a) a tumor cell;
(b) a reactive tissue; and
(c) a liquid carrier solution, wherein the composition is succussed to a homeopathic potency of at least about 200 c.

19. The homeopathic composition of claim 18 wherein said tumor is selected from the group consisting of a soft tissue sarcoma, a lymphoma, a brain tumor, an esophagus tumor, a uterine cervix tumor, a bone tumor, a lung tumor, an endometrial tumor, a bladder tumor, a breast tumor, a larynx tumor, a colon/rectum tumor, a stomach tumor, an ovary tumor, a pancreas tumor, an adrenal gland tumor, and a prostate tumor.

20. The homeopathic composition of claim 18 wherein said reactive tissue is selected from the group consisting of a monocyte, a T cell, a cytokine, a cell membrane, an inflammatory cell, and a mediator.

21. The homeopathic composition of claim 18 wherein said liquid carrier solution comprises at least 60% EtOH in water (volume to volume).

22. The homeopathic composition of claim 18 wherein said homeopathic composition has a potency of at least 200 c.

23. The homeopathic composition of claim 18 wherein said homeopathic composition has a potency of at least 1000 c.

24. The homeopathic composition of claim 18 further comprising a second homeopathic composition.

25. The homeopathic composition of claim 24 wherein said second homeopathic composition is selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium.

26. The homeopathic composition of claim 25 wherein the potency of said second homeopathic composition is added to a homeopathic potency of at least 200 c.

27. A method for prolonging the survival of an animal afflicted with a cancer, said method comprising the step of administering to said animal a homeopathic composition of any one of claim 8 or claim 18.

28. The method of claim 27 wherein said animal is a human.

29. The method of claim 27 wherein said homeopathic composition is administered at least twice.

30. The method of claim 27 wherein said homeopathic composition is administered to said animal at least once weekly for a period of at least one four weeks.

31. The method of claim 27 further comprising the step of administering a second homeopathic composition.

32. The method of claim 31 wherein said second homeopathic composition is selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium.

33. The method of claim 31 wherein said second homeopathic composition is added to a homeopathic potency of at least 200 c.

34. The method of claim 33 wherein said homeopathic composition is administered at least once weekly and wherein said second homeopathic composition is administered at least once weekly.

35. The method of claim 33 wherein said homeopathic composition is administered at least once weekly and wherein said second homeopathic composition is administered at least twice weekly.

36. A method for the treatment of a cancer in a patient in need thereof, said method comprising the step of administering to said patient a homeopathic composition of any one of claim 8 or claim 18.

37. The method of claim 36 wherein said homeopathic composition is administered at least twice.

38. The method of claim 36 wherein said homeopathic composition is administered to said animal at least once weekly for a period of at least one four weeks.

39. The method of claim 36 further comprising the step of administering a second homeopathic composition.

40. The method of claim 39 wherein said second homeopathic composition is selected from the group consisting of: Brain Cancer: Plumbum iodatum, Baryta carbonica, Aethusa cynapium, Zincum sulphuricum, Oral Cavity: Aurum muriaticu, hydrastis, Larynx and Vocal chord: Thuja, Agentum nitrcum, Lachesis, Kali bichromium, Thyroid and Parotid gland: Thuja, Spongia, Iodum, Lachesis, Esophagus: Causticum, Thuja, Argentum nitricum, Silica, Arsenicum album, Breast: Conium, Pulsatilla, Sepia, Phytolacca, Phosphorus, Stomach: Hydrastis, Cadmium sulphuricum, Arsenic album, Kali bichromicum, Ornthogallum, Pancreas: Hydrastis, Ceanothus americannus, Arsenic album, Cadmium sulphuricum, Baryta iodata, Liver: Chelidonium, Hydrastis, Lycopodium, Natrum Sulphuricum, Colon: Aloe, Thuja, Arsenicum album, Lycopodium, Rectum: Nitricum acidum, Aloe, Thuja, Lachesis, Sulphur, Bladder: Terbinthina, Thuja, Conium, Prostate: Thuja, Conium, Sabal serrulata, Lycopodium, Ovaries: Aurum muriaticum natronatum, Viburnum prunifolium, Thuja, Sepia, Lilium tigrinum, Uterus: Aurum muriaticum natronatum, Viburnum prunifolium, Thuja, Sepia, Lilium tigrinum, Lachesis, Cervix: Aurum muriaticum natronatum, Pulsatilla, Sepia, Lilium tigrinum, Bone: Hekla lava, Symphytum, Ceanothus americanus, Strontia, Hodgkin's lymphoma: Arum mettalicum. Iodum, Spogia, Acrophularia nodosa, Thuja, Melanoma: Arsenicum album, Arsenicum bromatum, Causticum, Calcarea arsenica, and Skin: Arsenicum bromatum, Arsenicum album, Sulphur, Euphorbium.

41. The method of claim 40 wherein said second homeopathic composition is added to a homeopathic potency of at least 200 c.

42. The method of claim 40 wherein said homeopathic composition is administered at least once weekly and wherein said second homeopathic composition is administered at least once weekly.

43. The method of claim 40 wherein said homeopathic composition is administered at least once weekly and wherein said second homeopathic composition is administered at least twice weekly.
